(19) United States
(12) Patent Application Publication
(10) Pub. No.: US 2010/0204712 A1
(43) Pub. Date: Aug. 12, 2010

(54) NEUROVASCULAR MICROCATHETER DEVICE, SYSTEM AND METHODS FOR USE THEREOF

(76) Inventor: Mark Mallaby, San Diego, CA (US)

Correspondence Address: The Law Office of Jane K. Babin;Professional Corporation, c/o Intellevate, P.O. Box 52050, Minneapolis, MN, 55402, US

(21) Appl. No.: 12/704,492

(22) Filed: Feb. 11, 2010

Related U.S. Application Data
(60) Provisional application No. 61/151,678, filed on Feb. 11, 2009.

(51) Int. Cl. A61B 17/22

(52) U.S. Cl. 606/128

(57) ABSTRACT

The present invention relates to neurovascular thrombectomy devices that include an occlusion microcatheter and fluid-assisted microcatheter fitted within the occlusion microcatheter. Also provided are systems and methods for neurovascular thrombectomy using the devices of the invention.

### RELATED APPLICATIONS

This application claims the benefit of priority under 35 USC §119 of U.S. Provisional Applications Ser. No. 61/151,678 filed Feb. 11, 2009, the entire disclosure of which is incorporated herein by reference.

### FIELD OF THE INVENTION

The present invention relates generally to neurovascular catheters. Specifically, the invention relates to microcatheters and more particularly fluid jet assisted microcatheter systems for removal of organized and non-organized thrombetic material from occluded blood vessels.

### BACKGROUND OF THE INVENTION

Thrombetic material occlusion or blood clots are the leading cause of neurological organ failure or brain damage, in the form of ischemic stroke, accounting for 85% of stroke incidents. More than 700,000 people in the U.S are affected by initial or recurrent ischemic stroke every year, with about 1 incident occurring every 45 seconds. The cost to treat stroke patients is over $56 billion dollars per year, and yet more than half of these patients die while the surviving patients may suffer long-term disability requiring continuing care and reducing quality of life.

Current treatment options for thrombetic occlusion include pharmaceutical intervention (e.g. administration of tissue plasminogen activators (tPAs)) and surgical thrombus removal strategies. For most patients (approximately 95%), tPAs are not a viable treatment due to clinical preconditions and delays in receiving treatment, which is limited to a three hour limited use window as the thrombetic burden becomes too organized for pharmaceutical treatment thereafter.

Surgical interventions utilize catheter-based devices for removing obstructions from small blood vessels including simple aspiration devices that pull thrombus into an evacuation tube; mechanical devices that utilize a wire, basket or coil to grab and extract thrombus; and devices that combine aspiration with a mechanical element. Fluid-assisted thrombus removal devices spray jets of saline or pharmaceutical formulations to facilitate thrombus removal while ultrasonic devices disrupt occluding thrombus using sound waves. Each of these types of devices is limited in effectiveness for various situations and involves inherent risks associated with the particular design.

Aspiration Devices. Simple aspiration devices are effective in removing only the most acute levels of thrombus and they do not extend the treatable period for ischemic stroke beyond the current pharmaceutical options. Simple aspiration devices tend to become blocked with thrombus which limits their effectiveness and can create treatment complications. Generally, simple aspiration catheters are limited to use in larger vessels not normally associated with neurovascular treatment.

Mechanical Disruption Devices. Mechanical thrombus removal devices use a physical capture element (e.g., wire, coil, basket) to contain thrombetic material for removal. Such devices are more suitable for removing organized thrombus than aspiration devices, thereby extending the time these devices can be used in stroke intervention strategies.

The primary limitation of mechanically-assisted devices is that the thrombus capture element needs to be deployed distally to the occluding thrombus. The act of passing the capture mechanism through and beyond the occluding thrombus will often break thrombetic material away from the primary thrombus site. Uncontrolled disruption risks distal embolism formation in the brain, lungs and other vital organs as thrombetic fragments travel within the circulation and may lodge in distant sites. Moreover, mechanical devices typically contact vessel walls, which can result in hemodynamic stress, intimal damage to the vessel wall, and in worst cases vessel perforation leading to hemorrhagic ischemia.

Ultrasound Devices. Ultrasound technology has also been used to open blocked or restricted arteries. However, ultrasonic devices are expensive, bulky and must also cross the thrombus target site, risking dislodging emboli as described above. The distal end of ultrasound devices has a relatively large profile, making them unsuitable for maneuvering through tortuous vasculature, such as found in the brain.

Fluid-Assisted Devices. Devices that use jets or sprays of saline or other fluids to dislodge thrombus can deliver the fluid within the catheter (internal devices) or dispense it from the catheter into the vessel (external devices). External devices that spray saline outward to dislodge thrombus from the vessel wall are inherently unsuitable for use in the small vessels of the neurologic field because they risk hemodynamic stress and intimal damage to the vessel wall. Internal jets can be used to emulsify or macerate occluding thrombus, for example, by providing a back-spray that is advanced beyond the thrombus and forces thrombetic material into the device catheter. Such devices can be complicated and bulky, and thus have limited applicability and effectiveness in neurovascular situations.

In addition, the overall instrument and procedural design must be contemplated for neurovascular surgical intervention as the catheter alone is not useful. In order to treat neurovascular regions for ischemic stroke it is often desirable to restrict blood flow immediately proximal to the occluding thrombus. Although systems and devices are available to perform this task, they are also limited by design drawbacks that can cause clinical complications. When blood flow restriction is available, the system must also assess and carefully control the method and rate at which reperfusion occurs following the procedure. If the reperfusion of the treated area is too rapid, vessel hemorrhage or further ischemic events may occur.

Thus, there is a continued need in the art for improved thrombus removal devices and methods that can be safely used in the neurovasculature to effectively remove blood clots without leading to serious complications.

### SUMMARY OF THE INVENTION

The present invention provides a neurovascular thrombectomy device that includes a generally tubular, hollow occlusion microcatheter having a distal end adapted for vascular occlusion and a proximal end adapted for controlling said vascular occlusion; and a generally tubular fluid-assisted microcatheter fitted within said occlusion microcatheter having a distal end adapted for fluid-assisted thrombus removal and a proximal end adapted for controlling said fluid-assisted thrombus removal.

The fluid-assisted microcatheter is a generally hollow tubular body defining an evacuation path, where the hollow body includes an inner lumen and an outer lumen defining a sealed fluid delivery intra-luminal space. The fluid-assisted microcatheter can, for example, be slidably fitted within the occlusion microcatheter.

In certain embodiments, the neurovascular thrombectomy device includes a fluid access port in fluid communication with the fluid delivery intra-luminal space; and a fluid jet orifice disposed near the distal end of the inner lumen, which can deliver fluid under high pressure through the fluid delivery intra-luminal space to the fluid jet orifice and into the evacuation path.

In certain embodiments, the open distal end of the fluid-assisted microcatheter is generally perpendicular to the length of the neurovascular thrombectomy device, thereby forming a round aspiration orifice. In other embodiments, the distal end of the fluid-assisted microcatheter is angled 45 degrees relative to the length of the neurovascular thrombectomy device thereby forming an oval aspiration orifice. In certain of such embodiments, the fluid jet orifice can deliver fluid directly into the oval aspiration orifice when it is located.

The occlusion microcatheter will typically include an occlusion balloon and may have a haemostatic seal in an opening at its proximal end.

The occlusion microcatheter will generally include an inner tubular member surrounding a working channel and an outer tubular member surrounding the inner tubular member, said outer tubular member and said inner tubular member together defining an inflation intra-luminal space. Typically, the working channel has a diameter sufficient to slidably accept the fluid-assisted microcatheter.

In certain embodiments, the occlusion microcatheter includes an inflation opening disposed on the outer tubular member, and an balloon surrounds the inflation opening and is sealably secured to the outer tubular member, such that the balloon is in fluid communication with the inflation intra-luminal space through the inflation opening. In other embodiments, the balloon is formed as a contiguous outpocketing of the outer tubular member. In yet further embodiments, the balloon sealably bridges the outer tubular member and the inner tubular member. In certain aspects, the inner tubular member is longer than and extends distally beyond the outer tubular member to provide a narrower entry profile for the occlusion microcatheter.

At its proximal end, the occlusion microcatheter is optionally sealed with a haemostatic seal and adapted for controlling vascular occlusion, which may be accomplished using a three-way reperfusion valve. Optionally, the three-way reperfusion valve is detachably disposed on the proximal end of the proximal end of the occlusion microcatheter.

The present invention also provides a neurovascular thrombectomy system that includes the neurovascular thrombectomy device of the invention and may also include: a fluid reservoir connected to the fluid access port; a high pressure fluid delivery means for conveying fluid from the fluid reservoir to the fluid access port; an aspiration means connected to the aspiration exit port; and/or an aspiration receptacle connected in-line between the aspiration means and the occlusion microcatheter for receiving thrombus and fluid.

The present invention also provides methods for treating neurovascular thrombus including the steps of: a) accessing a patient blood vessel using a Seldinger technique; b) inserting a guide wire through the accessed blood vessel and maneuvering the guide wire to a treatment site; c) placing a guide catheter over the guide wire and advancing the guide catheter to a position slightly proximal to the treatment site; d) inserting the occlusion microcatheter of the neurovascular thrombectomy system of the invention into the guide catheter and advancing the occlusion microcatheter to the treatment site; e) inserting the fluid-assisted microcatheter of the invention over the guide wire, into the occlusion microcatheter and sliding the fluid-assisted microcatheter to the treatment site; f) removing the guide wire; g) inflating the occlusion balloon, thereby occluding blood flow proximal to the treatment site; h) simultaneously activating the high pressure fluid delivery means and the aspiration means, thereby powering the fluid-assisted microcatheter of the neurovascular thrombectomy system; i) advancing the powered fluid-assisted microcatheter fluid to or through a thrombus at the treatment site, thereby removing thrombus; j) repeating step i) until all thrombus is removed from the treatment site; k) simultaneously deactivating the high pressure fluid delivery means and the aspiration means; l) withdrawing the fluid-assisted microcatheter from the treatment site; m) restoring blood flow to the treatment site by deflating the occlusion balloon; n) removing the fluid-assisted microcatheter, occlusion microcatheter and guide catheter from the patient, thereby removing neurovascular thrombus. Optionally, the method uses a three-way reperfusion valve adapted for controllably inflating and deflating the occlusion balloon.

### BRIEF DESCRIPTION OF THE DRAWINGS

FIG. 1 is an overall schematic drawing of a neurovascular thrombectomy microcatheter system.

FIG. 2 is a longitudinal section view of a fluid-assisted aspiration microcatheter 104 and multi-access hub 70 according to one embodiment of the invention.

FIG. 3 is a cross sectional view of the distal tip 30 of a fluid-assisted aspiration microcatheter 104 of an embodiment of the invention having a tapered tip.

FIG. 4 is a cross sectional view of the distal tip 30 of a fluid-assisted microcatheter 104 of an embodiment of the invention having a soft tapered tip.

FIG. 5 shows a longitudinal section of the distal region of a neurovascular thrombectomy device within a treatment site in a patient cranial vasculature 12, showing the placement of fluid-assisted aspiration microcatheter 104 relative to occlusion microcatheter 102.

FIG. 6 is a detailed longitudinal section view illustrating an embodiment of occlusion microcatheter 102 equipped with an in-line occlusion balloon 26 (shown inflated).

FIG. 7 is a detailed longitudinal section view of occlusion microcatheter 102 equipped with an occlusion balloon 26 (shown inflated).

FIGS. 8A and 8B show side and front views, respectively, of three-way reperfusion valve 33 in an “off” position.

FIGS. 9A and 9B show side and front views, respectively, of three-way reperfusion valve 33 in a “reperfuse” position.

FIGS. 10A and 10B show side and front views, respectively, of three-way reperfusion valve 33 in a “full flow” position.

The many advantages of the present invention will be apparent to those skilled in the art with the reading of this specification in conjunction with the enclosed drawings wherein like reference numerals are applied to like elements and wherein.

### DETAILED DESCRIPTION

It is to be understood that both the foregoing general description and the following detailed description are exemplary and explanatory only and are not restrictive of the invention claimed. As used herein, the use of the singular includes the plural unless specifically stated otherwise. As used herein, “or” means “and/or” unless stated otherwise. Furthermore, use of the term “including” as well as other forms, such as “includes,” and “included,” is not limiting. The section headings used herein are for organizational purposes only and are not to be construed as limiting the subject matter described.

Unless specific definitions are provided, the nomenclatures utilized in connection with, and the laboratory procedures and techniques of neurology, vascular biology, neurosurgery and general medicine described herein are those known in the art. Standard symbols are used interchangeably with the full names represented by such symbols. Thus, for example, the terms “Fr” and “French” are understood to have identical meaning Standard techniques may be used for surgical procedures, manufacturing of medical devices and.

### Definitions

“About” as used herein means that a number referred to as “about” comprises the recited number plus or minus 1-10% of that recited number. For example, “about” 100 cm can mean 95-105 cm or as few as 99-101 cm depending on the situation. Whenever it appears herein, a numerical range such as “1 to 20” refers to cm to each integer in the given range; e.g., “1 to 20 cm” means that an a catheter be only 1 cm in length, 2 cm in length, 3 cm in length, etc., up to and including 20 cm in length.

“Vasculature” refers to the network of blood vessels of an organ or body part, including arteries, capillaries and veins. “Neurovasculature”is used herein to refer to blood vessels associated with the nervous system, and in particular, those within, supplying or returning blood to or from the brain.

As used herein, “thrombus” refers to an aggregation of blood factors, primarily platelets and fibrin with entrapment of cellular elements, frequently causing vascular obstruction at the point of its formation or restrictive capture. Most ischemic events are from coronary thrombus. Newly formed (“fresh” or “unorganized”) thrombus is generally soft and malleable. As thrombus ages, it becomes more rigid or “organized” and is more difficult to remove from the vasculature.

As used herein, “embolism” refers to the sudden blocking of an artery by a thrombus or foreign material.

### Neurovascular Thrombectomy Device

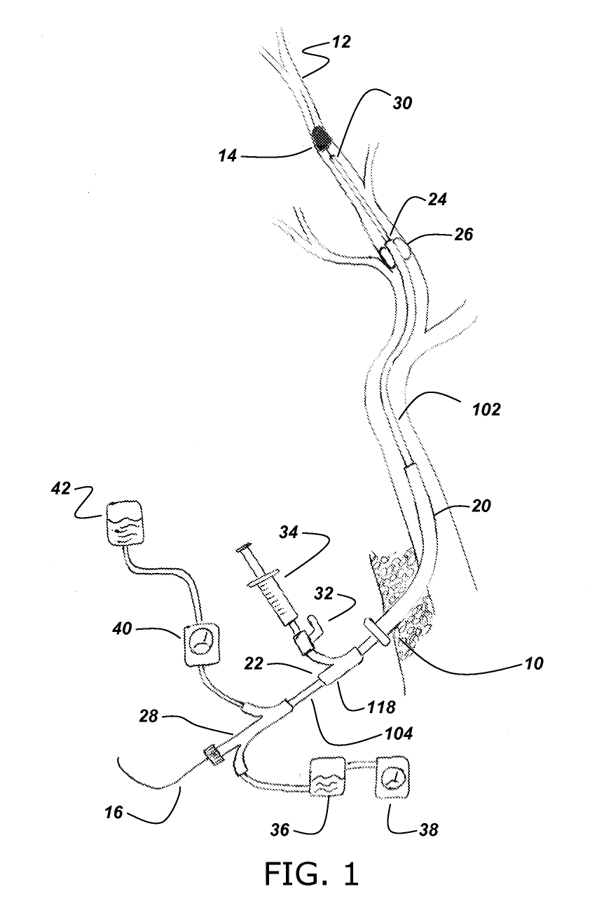

Referring to the drawings in detail, wherein like numerals indicate like elements, and specifically to FIG. 1, the present invention provides a neurovascular thrombectomy device, shown in the context of a complete thrombectomy system, that includes a fluid-assisted aspiration microcatheter 104 fitted within an occlusion microcatheter 102. In order to maximize the cross-sectional area, components of the neurovascular thrombectomy device are generally tubular in shape, the occlusion microcatheter 102 having a generally larger diameter than the fluid-assisted aspiration microcatheter 104. In operation, the occlusion microcatheter 102 component of the device provides proximal blood flow restriction of the neurovasculature surrounding the treatment site, while the fluid-assisted aspiration microcatheter component 104 is adapted for removing thrombus. As used herein, “treatment site” refers the site of a thrombus in the neurovasculature of a patient and the area immediately surrounding the thrombus.

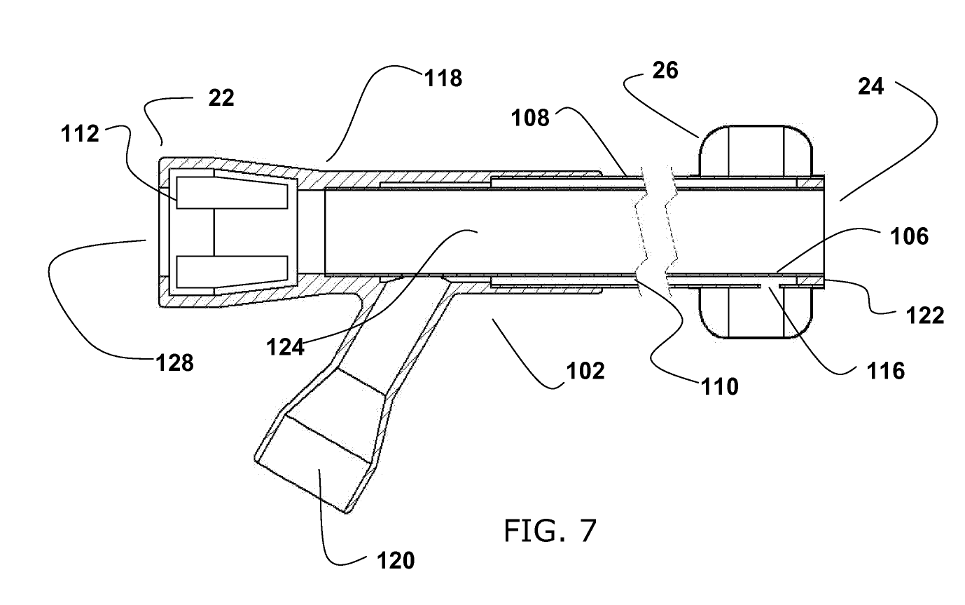

The occlusion microcatheter 102 is generally a hollow, tubular body defining a working channel 124 (identified e.g. in FIG. 7), having a proximal end 22 adapted for controlling an occlusion mechanism (such as an occlusion balloon as shown 26) and a distal end 24 adapted to occlude a blood vessel. The skilled artisan will be aware of a variety of occlusion mechanisms suitable for use in the neurovascular thrombectomy device of the present invention. In one embodiment, the occlusion microcatheter 102 includes an occlusion balloon 26 adapted to reversibly and controllably restrict blood flow through a vessel of the neurovasculature. During insertion of the occlusion microcatheter 102 into the neurovasculature, the occlusion balloon 26 is deflated to allow ease of passage within the blood vessel. Once the occlusion microcatheter 102 has been advanced to a position near the site of a thrombus, the occlusion balloon 26 can be inflated until blood flow surrounding the balloon is stopped. With blood flood stopped, the fluid-assisted aspiration microcatheter 104 within the occlusion microcatheter 102 can safely be operated to remove thrombus 14 by aspiration and/or fluid-assisted aspiration as necessary. By restricting blood flow around a treatment site, repeated cycles of fluid application followed by thrombus aspiration are possible without the danger of the thrombus (or a portion thereof) traveling from site.

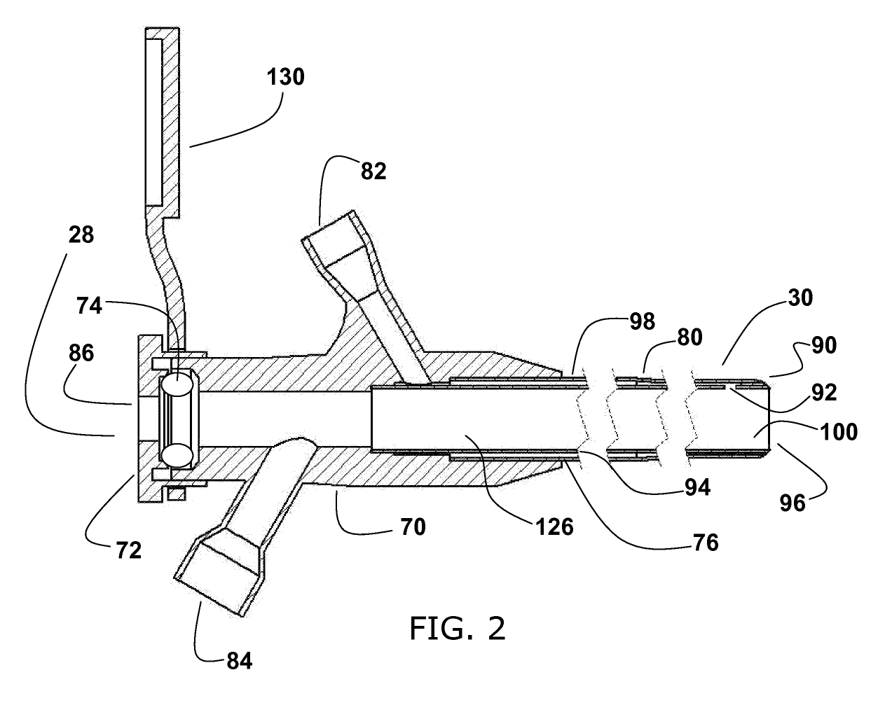

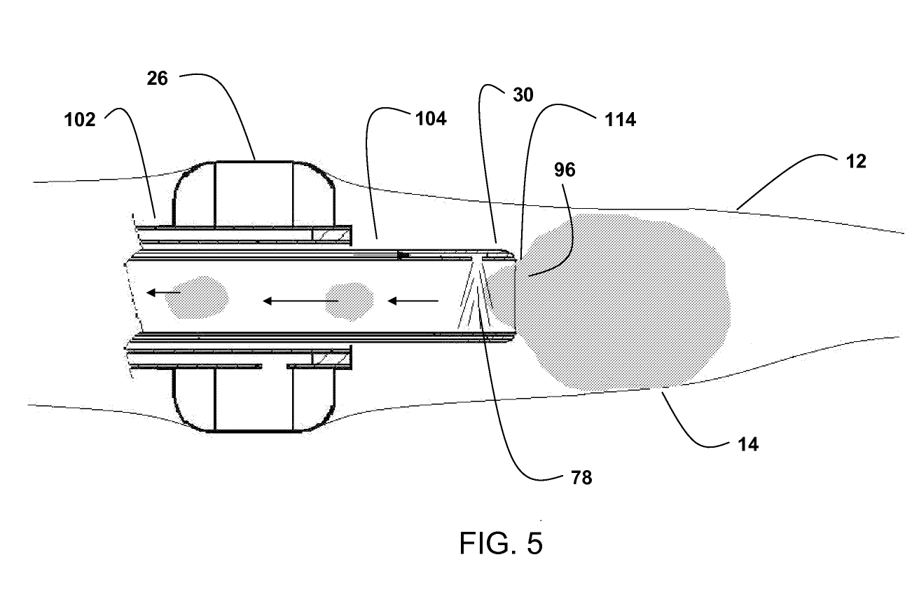

The fluid-assisted aspiration microcatheter 104 (illustrated e.g. in FIG. 2 and FIG. 5) is a generally hollow, tubular body defining an evacuation path 126 (shown directionally in FIG. 5 by arrows), having a proximal end 28 adapted for controlling both intravascular fluid application and aspiration of fluid and thrombus; and a main body or shaft 76 that terminates at a distal end 30 that is adapted to apply fluid within evacuation path 126, and to accept thrombus and applied fluid through the evacuation path 126 upon application of suction via the proximal controls.

In certain embodiments of the invention, fluid-assisted aspiration microcatheter 104 is fitted snugly but slidably within the working channel 124 of occlusion microcatheter 102 as shown in FIG. 2. The distal end of fluid-assisted aspiration microcatheter 104 can be advanced to extend beyond the distal end of occlusion microcatheter 102 (FIG. 5). Providing a slidable aspiration microcatheter-occlusion microcatheter assembly permits the occlusion microcatheter 102 to first be advanced to a treatment site position near, but somewhat proximal to a thrombus. The occlusion mechanism can then be fully or partially activated (e.g., a balloon mechanism 26 inflated) to secure the occlusion microcatheter at the desired location and/or restrict blood flow surrounding the treatment site. Once the occlusion microcatheter is positioned, the fluid-assisted aspiration microcatheter 104 can be advanced through the working channel 124 of occlusion microcatheter 102 to the position of the thrombus. In such embodiments, the distal end 30 of fluid-assisted aspiration microcatheter 104 typically exits the distal end 24 of occlusion microcatheter 102, extending therethrough to a position at or near a thrombus. This configuration of the neurovascular thrombectomy device not only permits independent positioning of the occlusion microcatheter 102 and fluid-assisted aspiration microcatheter 104, it also allows the operator to restrict blood flow around the treatment site prior to positioning the distal end 30 of fluid-assisted aspiration microcatheter 104, and/or to reposition fluid-assisted aspiration microcatheter 104 as needed during thrombectomy.

In certain aspects of the invention, and particularly where the occlusion microcatheter 102 includes a balloon, inflating the balloon, in conjunction with the arrangement of fluid-assisted aspiration microcatheter 104 within occlusion microcatheter 102, physically isolates and protects the surrounding vasculature from inadvertent contact with fluid-assisted aspiration microcatheter 104 before and during thrombus removal. Specifically, the fluid-assisted aspiration microcatheter 104, by fitting within and thus having a narrower diameter than occlusion microcatheter 102, is separated from the patient vessel wall by at least the thickness of the occlusion microcatheter 102. Therefore the occlusion microcatheter 102 provides a barrier between fluid-assisted aspiration microcatheter 104 and the vessel wall. Inflation of occlusion balloon 26 expands the vasculature near the treatment site, further distancing the distal end 30 of fluid-assisted aspiration microcatheter 104 from the vessel wall.

### Occlusion Microcatheter

Turning to occlusion microcatheter 102 as shown generally in FIG. 1, the occlusion microcatheter and any parts thereof, can be of any construction and of any material suitable for surgical instruments. Generally, the materials and construction of all components of the neurovascular thrombectomy device, such as occlusion microcatheter 102, will provide sufficient strength, kink resistance and biocompatibility for operation in a human patient as described herein. Suitable materials for manufacturing the devices and systems of the invention are well known in the art.

The occlusion microcatheter 102 can be of any length sufficient to be advanced from a peripheral vein or artery to the desired treatment site. The occlusion microcatheter 102 will typically have a length of about 45 cm to about 170 cm, often a length of about 80 cm to about 140 cm, and frequently will be about 110 cm in length. Non-limiting examples of suitable lengths for occlusion microcatheter 102 include about 50 cm, about 60 cm, about 70 cm, about 80 cm, about 90 cm, about 100 cm, about 110 cm, about 120 cm, about 130 cm, about 140 cm, about 150 cm, about 160 cm, and about 170 cm. In certain embodiments of the invention, even longer catheter lengths are contemplated to facilitate operation by robotics, electronic controls or remote operation. The occlusion microcatheter 102 is of a sufficient diameter to be mechanically and clinically compatible with introducer sheaths or guide catheters 20 ranging from 4 Fr to 9 Fr. Thus, the outer diameter of the occlusion microcatheter 102 will typically be less than about 4 Fr to about 8.9 Fr. However, only dimensions suitable for use in the contemplated treatment sites within the neurovasculature are included in the scope of this invention.

In certain embodiments, occlusion microcatheter 102 is reusable and is made from durable materials known in the art that can be cleaned and sterilized prior to use. In other embodiments, the occlusion microcatheter 102 is intended for single use (i.e. is disposable) and is provided pre-sterilized.

### Operation of Neurovascular Thrombectomy Device

In operation, it is contemplated that occlusion microcatheter 102 will be inserted into the vasculature using a Seldinger or similar technique (although any suitable procedure for accessing the neurovasculature that is known in the art may be used for insertion of occlusion microcatheter 102). Briefly, the desired blood vessel is entered at a vascular access site 10 (e.g. an accessible peripheral artery) using a sharp, hollow needle (trocar; not shown). The vascular access site can be, for example, a region of the subclavian artery or femoral artery, or any accessible blood vessel from which the treatment site can be approached. A guide wire 16 is then advanced through the hollow lumen of the trocar under fluoroscopic guidance, and the trocar is withdrawn. An introducer sheath or guide catheter 20 is then passed over the guide wire 16 into the vessel and advanced to the treatment site where it is positioned in a manner permitting insertion of a microcatheter, such as the neurovascular thrombectomy device of the present invention, or other devices. Upon completion of the desired procedure, the guide catheter 20 and neurovascular thrombectomy device are withdrawn. The neurovascular thrombectomy device of the invention can thus be placed and operated at a treatment site within the neurovasculature. In certain embodiments, occlusion catheter 102 is permanently fitted within or is an integral part of the introducer or guide catheter 20.

In use, the proximal end 22 of occlusion microcatheter 102 is retained outside the patient's body, and is adapted for reversibly controlling vascular occlusion. Such adaptation may, for example, include a stopcock or valve 32, which can be three-way reperfusion valve 33 or any stopcock, valve or other means known in the art for controlling fluid flow, and/or a balloon inflation syringe 34 in fluid communication with an occlusion balloon 26, thereby permitting the operator to controllably inflate the occlusion balloon 26. The skilled artisan will be knowledgeable of additional occlusion devices and control mechanisms suitable for use with the present invention.

FIG. 7 illustrates an exemplary embodiment of an occlusion microcatheter 102 according to the invention, having a proximal end 22 and a distal end 24. This figure shows a longitudinal section view of the occlusion microcatheter 102, which is generally a hollow tubular body defining a path (working channel 124) for accessing a vascular treatment site. In certain embodiments, the occlusion microcatheter 102 includes an inner tubular member 106 surrounding the working channel 124, and an outer tubular member 108 surrounding the inner tubular member 106. The inner (106) and outer (108) tubular members together define an inflation intra-luminal space 110, which is a generally hollow, sealed space lying between the tubular members (106 and 108), through which inflation fluid can flow. According to the embodiment of the invention illustrated in FIG. 7, an inflation opening 116 is disposed on the distal end of the outer tubular member 108. Surrounding inflation opening 116, an occlusion balloon 26 is bonded, melted or otherwise sealably secured to the outer tubular member 108, thereby providing fluid communication between the occlusion balloon 26 and inflation intra-luminal space 110, whereby the balloon can be reversible inflated to provide vascular occlusion. In other embodiments, the outer tubular member and occlusion balloon 26 are fabricated, molded or otherwise manufactured as a single tubular member. In such embodiments, outer tubular member 108, has disposed thereon a contiguous pouch or out pocket that serves as the occlusion balloon 26. Whether formed as an integral part of the outer tubular member geometry or disposed upon the outer tubular member, inflation opening 116 is of sufficient size as not to limit overall inflation fluid flow rate within the inflation intra-luminal space 110. The distal end of the inflation intra-luminal space 110 is sealed by an adhesive plug 122 or other suitable seal or closure that will be well known in the art.

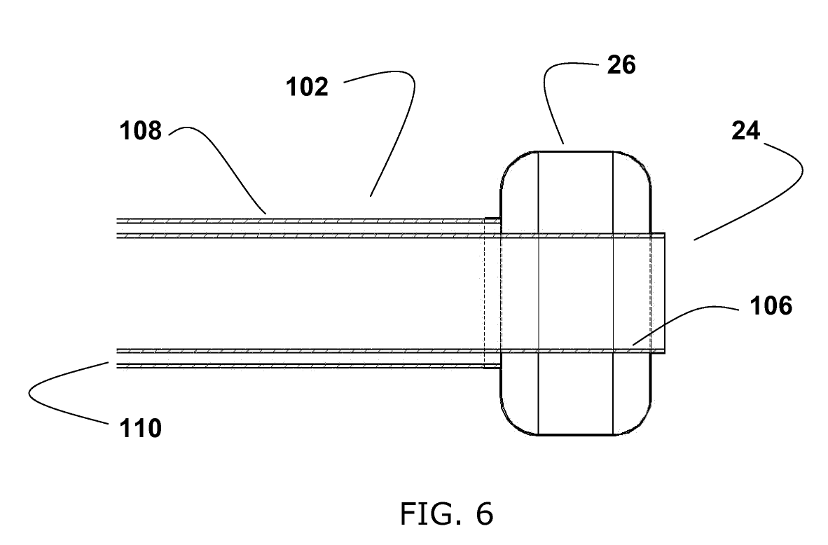

In certain embodiments, exemplified by the illustration of the distal end 24 of occlusion microcatheter 102 shown in FIG. 6, inner tubular member 106 is longer than outer tubular member 108, and thus extends distally beyond the outer tubular member 108 at distal end 24 of occlusion microcatheter 102. According to such embodiments, the extension of inner tubular member 106 creates a step between inner tubular member 106 and outer tubular member 108 that is bridged by occlusion balloon 26. The occlusion balloon 26 according to this embodiment has two edges or openings, such that a first edge or opening is bonded, melted or otherwise sealably secured at or proximal to the distal end of outer tubular member 108, and the second edge or opening is bonded, melted or otherwise sealably secured at or distal to the distal end of inner tubular member 106, thereby providing sealed fluid communication between the occlusion balloon 26 and inflation intra-luminal space 110. Such balloons can be reversibly inflated to provide vascular occlusion. In these embodiments the outer diameter of the distal end 24 of occlusion microcatheter 102 is tapered, thereby facilitating a lower entry profile for occlusion microcatheter 102 which enhances enhancing deliverability to the treatment site. The balloon itself forms the seal between inner tubular member 106 and outer tubular member 108, eliminating the need for an adhesive plug (shown as 122 in FIG. 7). In addition, the bridging function of the balloon in this embodiment establishes fluid communication between the balloon 26 and inflation intra-luminal space 110, thereby eliminating the need for inflation opening 116 (shown in FIG. 7).

In one embodiment of the invention, the proximal end 22 of occlusion microcatheter 102 includes a two-channel hub 118, that includes an inflation access port 120 adapted for controlling inflation fluid flow within the inflation intra-luminal space 110 and a proximal access port 128 that is in communication with working channel 124 through which fluid-assisted aspiration microcatheter 104 is slidably fitted. A haemostatic seal 112 is disposed on the inner tubular member at the proximal end 22 of occlusion microcatheter 102 to prevent blood or fluoroscopic fluids from escaping the vasculature through the proximal access port 128 during use.

The inflation access port 120 is adapted to receive inflation fluid, e.g., from a fluid filled inflation syringe 34. Thus, in one embodiment, inflation access port 120 terminates proximally in a luer lock type connector. The rate and amount of occlusion balloon 26 inflation is determined by the flow of inflation fluid received through the inflation access port 120, flowing through the inflation intra-luminal space and filling the occlusion balloon 26 via the intra-luminal inflation opening 116. Control of balloon inflation can be by any means available, such as manual or mechanical operation of the inflation syringe 34. Stopcocks and valves 32 suitable for controlling balloon inflation are known in the art. Following thrombectomy, the inflation process is reversed to deflate the balloon and allow reperfusion of the distal vasculature. The skilled artisan will recognize that controlled reperfusion is advantageous and may be required because rapid reperfusion within the delicate cranial vasculature can damage small vessel walls.

The present invention also provides an improved, three-way reperfusion valve 33 (described below), that facilitates superior control over balloon inflation and particularly reperfusion by preventing inadvertent and/or rapid deflation of occlusion balloon 26, and can be included used as stopcock or valve 32. Thus, in certain embodiments of the invention, inflation access port 120 is adapted to accept three-way reperfusion valve 33 for accurately controlling fluid flow. In another embodiment, three-way reperfusion valve 33 is disposed on the two-channel hub 118 as an integral part of inflation access port 120.

### Fluid-Assisted Aspiration Microcatheter

Turning to fluid-assisted aspiration microcatheter 104 as shown generally in FIG. 1, the fluid-assisted microcatheter and any parts thereof, can be of any construction and of any material suitable for surgical instruments. Generally, the materials and construction of all components of the neurovascular thrombectomy device, such as fluid-assisted aspiration microcatheter 104, will provide sufficient strength, kink resistance and biocompatibility for operation in a human patient as described herein.

In certain embodiments, fluid-assisted aspiration microcatheter 104 is reusable and is made from durable materials known in the art that can be cleaned and sterilized prior to use. In other embodiments, the fluid-assisted aspiration microcatheter 104 is intended for single use (i.e. is disposable) and can be provided in a pre-sterilized form.

The fluid-assisted aspiration microcatheter 104 is a generally a hollow, tubular body defining an evacuation path 126 (shown directionally in FIG. 5 by arrows), having a proximal end 28 adapted for controlling both fluid application and aspiration of fluid and thrombus; and a main body or shaft 76 that terminates at a distal end 30 that is adapted to apply fluid within the evacuation path 126, and to accept thrombus and applied fluid through evacuation path 126 upon application of suction via the proximal controls.

The fluid-assisted aspiration microcatheter 104 can be of any length sufficient to be advanced through the occlusion microcatheter 102 to the desired treatment site. The fluid-assisted aspiration microcatheter 104 will typically be longer than occlusion microcatheter 102 such that it can be advanced through and extend beyond occlusion microcatheter 102 when in use in the cranial vasculature. Furthermore, fluid-assisted aspiration microcatheter 104 will typically be longer than occlusion microcatheter 102 by a sufficient amount to permit proximal control of aspiration and fluid application, as illustrated in FIG. 1. Thus, fluid-assisted aspiration microcatheter 104 will typically have a length of about 60 cm to about 200 cm; often, a length of about 100 cm to about 160 cm, and frequently will be about 130 cm in length. Non-limiting examples of suitable lengths for fluid-assisted aspiration microcatheter 104 include about 60 cm, about 70 cm, about 80 cm, about 90 cm, about 100 cm, about 110 cm, about 120 cm, about 130 cm, about 140 cm, about 150 cm, about 160 cm, 170 cm, about 180 cm, about 190 cm, and about 200 cm. In certain embodiments of the invention, even longer catheter lengths are contemplated to facilitate operation by robotics, electronic controls or remote operation.

The fluid-assisted aspiration microcatheter 104 has a sufficient diameter to be mechanically compatible with and fit slidably within the working channel 124 of occlusion microcatheter 102. In certain embodiments, the main shaft 76 of fluid-assisted aspiration microcatheter 104 has an outer diameter ranging from less than about 3 Fr to about 8 Fr, typically about 3 Fr to about 7 Fr, and often about 3-4 Fr to about 6 Fr.

In certain aspects of the invention, space between fluid-assisted aspiration microcatheter 104 and occlusion microcatheter 102 is minimized to keep the overall size of the vascular thrombectomy device at a minimum, and is thereby only as large as absolutely necessary for sliding the fluid-assisted aspiration microcatheter 104 within working channel 124. In other embodiments, sufficient space remains in working channel 124 between the slidably fitted fluid-assisted aspiration microcatheter 104 and the inner tubular member of occlusion microcatheter 102 to allow injection of fluoroscopic fluid into the working channel 124 and permit the fluid to flow through the working channel to the distal end of the occlusion catheter 102 where it can diffuse into the vasculature at the treatment site. Injection of fluoroscopic fluid may thus be advantageously employed with certain neurovascular thrombectomy devices according to the invention, to enhance vascular visibility.

FIG. 2 provides a detailed longitudinal section view of fluid-assisted aspiration microcatheter 104, showing the generally hollow, tubular body defining evacuation path 126 through which fluid and thrombus are evacuated. In one embodiment of the invention, a multi-access hub 70 is disposed at the proximal end 28 of the fluid-assisted aspiration microcatheter 104 to facilitate control over aspiration of thrombus and fluid, e.g. including an aspirate exit port 84 in communication with the evacuation path 126. The multi-access hub 70 can also include fluid access port 82 adapted for controlling and applying fluid such as saline or another biocompatible fluid. The multi-access hub 70 also provides an independent channel for manipulating guide wire 16 within the evacuation path 126 while the device is being deployed at the treatment site. In this aspect, the invention includes a guide wire access port 86 that includes an adjustable (e.g. threaded) guide wire lock 72 that can be operated (e.g. turned clockwise) to compress a flexible bung 74, thus sealing and constricting the movement of the guide wire. In some embodiments, multi-access hub 70 can be covered by a flexible cap 130 that when fitted over the guide wire access port 86, seals the guide wire access port 86 to prevent aspirate material escaping or air entering through the evacuation path 126 during guide wire removal.

Distal to fluid access port 82, the main shaft 76 of the fluid-assisted aspiration microcatheter 104 is a bi-walled structure. The main shaft 76 includes an inner lumen 100 and an outer lumen 98 that together define a fluid delivery intra-luminal space 94 in fluid communication with the fluid access port 82 and fluid jet orifice 92. Fluid can be injected through fluid access port 82 into the fluid delivery intra-luminal space 94, which has a cross sectional area sufficient to allow a minimum fluid flow rate of about 4 ml/min and a maximum fluid flow rate of about 20 ml/min therethrough.

In certain embodiment, a taper 80 may be included on main shaft 76 such that the distal end 30 of the fluid-assisted aspiration microcatheter 104 has a smaller diameter than the more proximal regions. According to such embodiments, proximal to taper 80, main shaft 76 has a diameter that interfaces in an axial sliding motion with the occlusion microcatheter 102 (i.e. in working channel 124) without binding or kinking Distal to the taper 80, main shaft 76 narrows. The narrowing of such embodiments of the invention permits fluid-assisted aspiration microcatheter 104 to access very small neurovascular vessels (e.g. between about 1.0 mm and about 2.0 mm in diameter), even when more proximal regions of the fluid-assisted aspiration microcatheter 104 are of a larger diameter due to mechanical or manufacturing requirements. Taper 80 allows the use of a larger diameter for the main shaft 76 within the occlusion microcatheter 102, for example, to maintain high pressure within fluid delivery intra-luminal space 94 of the fluid-assisted aspiration microcatheter 104. The taper 80 can be as long as the manufacturing process allows and is located about 1.0 cm to about 145.0 cm from the distal end of fluid-assisted microcatheter 104.

In one exemplary embodiment, inner lumen 100 and outer lumen 98 are fused together other at the distal end of the fluid-assisted microcatheter 104 thereby sealing the fluid delivery intra-luminal space 94. According to this embodiment, the fluid-assisted microcatheter 104 thus terminates distally in a generally round aspiration orifice 96, which is generally perpendicular to main shaft 76 of fluid assisted microcatheter 104, for accepting thrombus from a treatment site. Optionally, a radiopaque marker band can be disposed at the distal end of the microcatheter 104 (e.g., within the fluid delivery intra-luminal space 94) to facilitate fluoroscopic localization of the fluid-assisted microcatheter 104 when inserted into the neurovasculature of a patient. In certain aspects of the invention the radiopaque marker band 90 acts together with adhesive or other means to seal the fluid delivery intra-luminal space 94 at the distal end of the microcatheter 104.

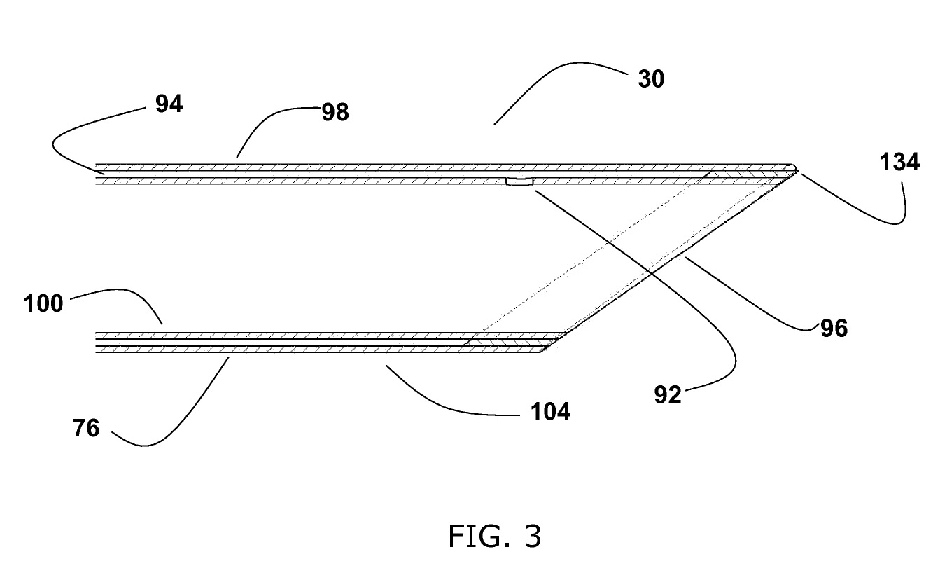

FIG. 3. illustrates another embodiment of the distal tip 30 of fluid assisted microcatheter 104 in which aspiration orifice 96 is cut, formed, molded or otherwise constructed at a about 15-80 degree angle, typically about 45 degrees, relative to main shaft 76 to form a generally oval shaped aspiration orifice 96. In this embodiment, fluid jet orifice 92 can be positioned more distally and closer to aspiration orifice 96.

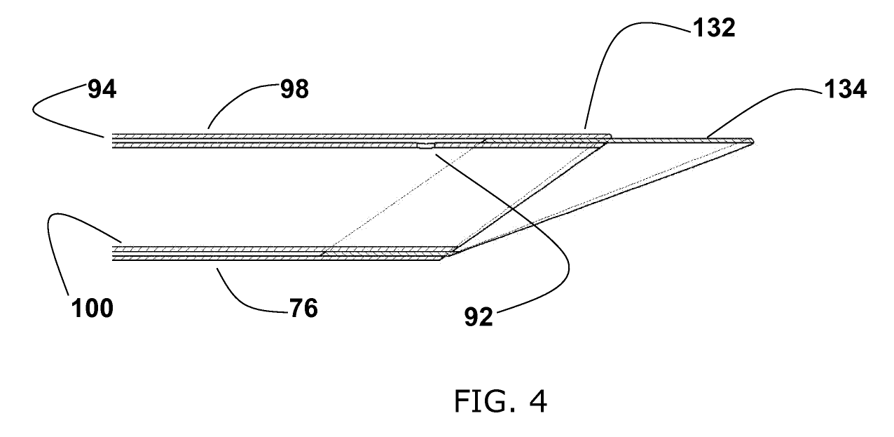

In yet other embodiments, as shown in FIG. 4, fluid assisted microcatheter 104 can be constructed to have a distal soft tip 134. In such embodiments, an angled distal plug 132 made of flexible material, is bonded melted or otherwise constructed to extend distally from main shaft 76 to form an atraumatic or soft distal tip. Such a soft distal tip is less likely to cause injury should it contact a vessel wall during use.

Inner lumen 100, outer lumen 98 and multi-access hub 70 can be constructed or fabricated from any material with sufficient strength to maintain functionality and resist kinking The radiopaque marker 90 band is constructed of a material that returns a visual indication to the operator under fluoroscopic guidance.

The fluid-assisted microcatheter 104 includes a fluid jet orifice 92 disposed at the distal end of the inner lumen 100, through which saline or other biocompatible fluid can be applied to a thrombus. The fluid jet orifice 92 is directed to spray fluid within the evacuation path 126 of the fluid-assisted microcatheter 104, rather than outward into the vasculature. Fluid jet orifice 92 is typically located within about 0.001 to about 0.150 inches from the distal end of the fluid-assisted microcatheter 104. The fluid jet orifice 92 is typically about 0.0003 to 0.0110 square inches in cross sectional area. Fluid jet pressure through fluid jet orifice 92 is about 50 to about 600 psi. The pressure can be controlled by any means known in the art, such as by providing a regulated, high-pressure fluid delivery means 40, such as a pump, and is calculated with the high pressure output and the ensuing system head loss.

In operation, fluid-assisted microcatheter 104 is advanced to the site of a thrombus and the thrombus is removed in a bimodal fashion. Upon contact, thrombus is drawn through aspiration orifice 96 toward aspiration exit port 84 by the application of suction to aspiration exit port 84 (e.g. via a vacuum means 38). Fresh, unorganized thrombus will typically be aspirated completely. However, more organized thrombus that is drawn into the aspiration orifice 96 may become lodged in the evacuation path 126, at or proximal to fluid jet orifice 92. When organized thrombus is present and becomes lodged, an internal fluid jet 78 can be applied through fluid jet orifice 92 to section or emulsify the lodged thrombus so that it can be aspirated and removed. Following initial thrombus removal, additional thrombus, if present, is evacuated through aspiration orifice 96 (e.g. by the suction force of vacuum means 38) thereby preventing loss of communication with thrombus. The aforementioned process can be continued until all occluding thrombus is removed.

The arrangement of fluid-assisted microcatheter 104 within occlusion microcatheter 102 coupled with the mechanical relationship between aspiration means 38, aspiration orifice 96, fluid jet orifice 92, and internal fluid jet 78 ensures that all emulsified, sectioned or otherwise dislodged thrombus is securely captured within the evacuation path 126 of the fluid-assisted microcatheter 104 without the distal tip of the microcatheter 104 contacting the intima of the vessel wall or allowing thrombus to re-enter the intracranial vasculature. In other words, thrombus removal from a treatment site is accomplished by negative pressure (i.e. suction) rather than positive pressure (e.g. grasping or scraping). When thrombus is too large or rigid to pass easily through the evacuation path, thrombus is reduced within the microcatheter device by fluid jet force rather than outside of the microcatheter device which could lead to further embolism or vascular damage. The neurothrombectomy device of the present invention thus provides consistently safe neurovascular thrombectomy, thereby reducing the risk of distal embolization, hemorrhage or vessel damage.

The fluid applied through fluid-assisted microcatheter 104 is typically saline or another physiologically compatible solution, but may include agents (i.e. pharmaceuticals or biologicals) that facilitate thrombus emulsification and/or liquefaction. Advantageously, the neurothrombectomy device may permit use of such agents even when contraindicated for administration locally or systemically to a patient. Because fluid is applied within the fluid-assisted microcatheter 104 and is continually aspirated from the evacuation path without contacting with the treatment site, the risk of adverse effects accompanying administration of the agent to the patient are eliminated.

### Three-Way Reperfusion Valve

The present invention also provides an improved, three-way reperfusion valve 33, for controlling occlusion balloon inflation, deflation and thereby vascular reperfusion. The three-way reperfusion valve 33 of the invention prevents rapid deflation of occlusion balloon 26.

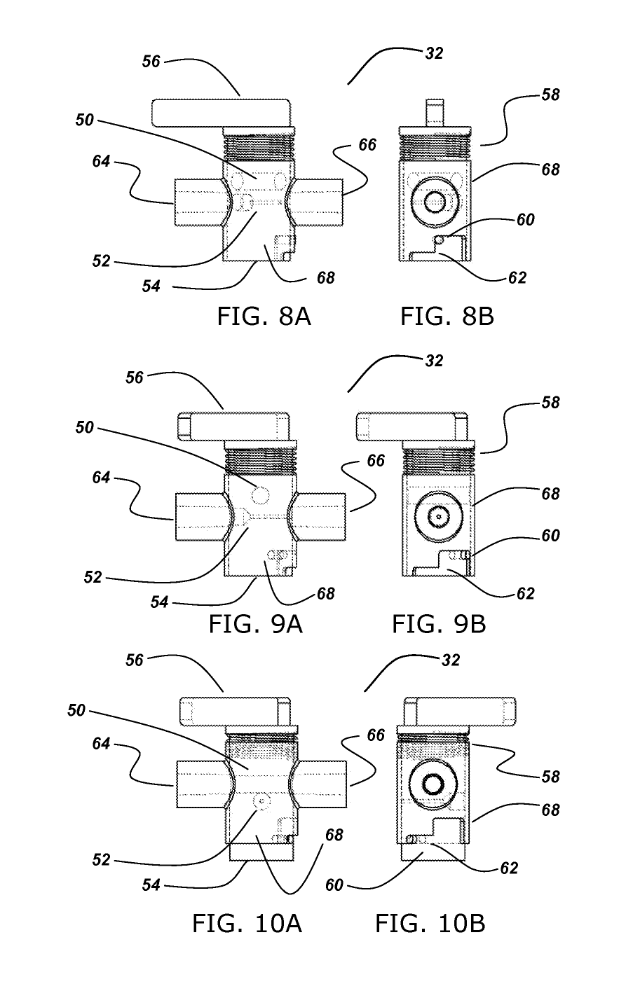

A nonlimiting embodiment of the three-way reperfusion valve of the invention is detailed in FIGS. 8A-10B, which illustrate side (A) and front (B) views of three-way reperfusion valve 33 in three operational positions. The three-way reperfusion valve 33 generally provides a controllable, bidirectional fluid path from a hollow, generally tubular first bidirectional fluid port 64, to a hollow, generally tubular second bidirectional fluid port 66, which ports are disposed on opposite sides of a generally tubular hollow valve body 68 and are of approximately the same diameter.

The three-way reperfusion valve 33 also includes a petcock 54 sealably seated within hollow valve body 68. The petcock 54 is generally a solid cylindrical body terminating in a small handle 56 or other mechanism for determining and adjusting the rotational position of the petcock 54 within the hollow valve body 68; the petcock 54 having two channels for fluid to flow therethrough when the petcock is seated in the body 68 and either channel is aligned with the first bidirectional fluid port 64 on one side and the second bidirectional fluid port 66 on the other side. The first channel in petcock 54 is a generally cylindrical, hollow full flow channel 50 having a diameter approximately equal to the diameter of first bidirectional fluid port 64 and second fluid port 66. The second channel in petcock 54 is a generally cylindrical, hollow reperfusion channel 52, having a smaller effective diameter than the diameter of either first bidirectional fluid port 64 or second bidirectional fluid port 66. In certain embodiments of the invention, the fluid flow axis of reperfusion channel 52 is oriented at approximately a 90° angle of rotation relative to the full flow channel and is physically separated from full flow channel 50 such that the two channels are not in fluid communication with each other. Rotation of the petcock allows the operator to align the bidirectional fluid ports 64 and 66 with either of the mutually exclusive channels (50 and 52), or with no channel.

Reperfusion channel 52 restricts fluid volume flowing therethrough to less that the amount flowing through full flow channel 50, for example, by having a smaller diameter than first bidirectional fluid port 64 and second bidirectional fluid port 66. The skilled artisan will be aware of addition mechanisms that may be suitable for restricting fluid flow through reperfusion channel 52 in the three-way reperfusion valve 33 of the invention, including for example narrowing orifices, needles and seats, fluid filters or any other means available to restrict fluid flow. When the petcock 54 is positioned such that the first bidirectional fluid port 64 and the second bidirectional fluid port 66 are not aligned with either full flow channel 50 or reperfusion channel 52, the valve is sealed, thereby preventing fluid flow therethrough. In this position, no flow is allowed between first and second bidirectional fluid ports 64 and 66.

Thus, three-way reperfusion valve 33 has three operating positions: off, full flow and reperfuse flow, corresponding to FIGS. 8A and 8B, FIGS. 9A and 9B, and FIGS. 10A and 10B, respectively. The skilled artisan will note that each of these 3 positions requires both a rotational alignment of the petcock 54 within the hollow valve body 68 and an “in” or “out” al alignment of petcock 54 within the hollow valve body 68.

In certain embodiments of the invention, hollow valve body 68 includes a detent 62 having 3 positions disposed in the wall thereof, and petcock 54 includes a detent pin 60 disposed thereon that is mated with detent 62, to restrict and facilitate positioning of petcock 54 to discrete “off,” “full flow” and “reperfuse” positions. In certain aspects, the three-way reperfusion valve 33 includes detent spring 58 surrounding petcock 54 to apply tension to petcock 54 and provide tactile feedback to the operator.

FIGS. 8A and 8B illustrate one embodiment of the three-way reperfusion valve 33 of the invention in the “off” position. In this position no fluid flow is permitted through the bidirectional fluid ports (64 and 66) via either the full flow channel 50 or the reperfusion channel 52. The handle 56 of petcock 54 is fixed relative to the full flow and reperfusion channels; thus the position of the petcock can be detected by the operator as a means for assessing the operational state of the valve. FIGS. 8A and 8B illustrate the “off” position having petcock handle 56 centered in-line with (parallel to) the first and second bidirectional fluid ports 64 and 66. The skilled artisan will appreciate that petcock 54 can also be constructed in a configuration such that the “off” position is selected when the petcock handle 56 is perpendicular to the first and second bidirectional fluid ports 64 and 66, or any position in between. Furthermore, detent pin 60 fits into a discrete position of detent 62. The discrete positioning of detent pin 60 in detent 62, together with tension from detent spring 58 provides a positive location for the “off” position using tactile feedback.

FIGS. 9A and 9B illustrate three-way reperfusion valve 33 of the invention in the “reperfuse” position. In this position, the petcock handle 56 is rotated counter clockwise from the axis of the first and second bidirectional fluid ports 64 and 66, such that communication is permitted between the first and second bidirectional fluid ports 64 and 66 through reperfusion channel 52. Reperfusion channel 52 limits fluid flow by creating a restriction through the reperfusion valve 33. In certain embodiments, the restriction results from a smaller overall diameter of reperfusion channel, 52, as described above. Detent pin 60 fits into a discrete reperfusion position of detent 62. The discrete positioning of detent pin 60 in detent 62 together with tension from detent spring 58 provide a positive location for the “reperfuse” position using tactile feedback.

FIGS. 10A and 10B show the three-way reperfusion valve 33 in the “full flow” position. To achieve this position, petcock 54 is inserted more deeply into hollow valve body 68, and then rotated (clockwise rotation shown) from the axis of the first and second bidirectional fluid ports (64 and 66), thereby aligning the fluid ports with full-flow channel 50 and permitting free fluid flow through the three-way reperfusion valve 33. Full flow operation can be used, for example, to prep the occlusion microcatheter 102 prior to use and to inflate occlusion balloon 26. Full fluid flow cannot be achieved using tactile feedback alone because detent pin 60 must be engaged in a position of detent 62 that requires significant force as well as rotation on petcock 54, while detent spring 58 creates resistance to that force. This feature of the valve creates a primary safety mechanism to prevent unintentional establishment of the full flow position.

### Neurovascular Microcatheter System

The present invention also provides a neurovascular microcatheter system for intracranial thrombus removal including the neurovascular thrombectomy device described herein; a fluid reservoir 42 adapted for connection to fluid access port 82 on multi access hub 70; a high pressure fluid delivery means 40 (such as a pump) for conveying fluid from the reservoir to the fluid access port; an aspiration means 38 (such as a vacuum pump) adapted for connection to aspiration exit port 84 on multi access hub 70; and an aspiration receptacle 36 adapted for connection in-line between the aspiration means 38 and the occlusion microcatheter 102, to receive thrombus and fluid aspirated from a treatment site.

The system may optionally include a sealed fluid delivery means to inflate occlusion balloon 26, which can be, for example, a fluid filled inflation syringe 34 adapted for connection to bidirectional fluid port 64 or 66 on a three-way reperfusion valve 33, which in turn is adapted for connection to inflation access port 120 disposed on the two-channel hub 118 of occlusion microcatheter 102.

### Methods for Removing Neurovascular Thrombus

The present invention also provides methods for removing thrombus from a neurovascular embolus including the steps of: introducing the occlusion microcatheter 102 of the neurovascular microcatheter device of the invention to a site proximal to a treatment site containing thrombus; restricting blood flow surrounding the treatment site by engaging an occlusion mechanism of the occlusion microcatheter 102; advancing the fluid-assisted microcatheter 104 of the neurovascular microcatheter device of the invention through the occlusion microcatheter 102 until the distal end of the fluid-assisted microcatheter 104 contacts the thrombus; aspirating thrombus through aspiration orifice 96, along the evacuation path 126 for removal; and reperfusing the treatment site by releasing the occlusion mechanism.

In certain embodiments, the occlusion microcatheter 102 and fluid-assisted microcatheter 104 are advanced over a guide wire 16, which is subsequently withdrawn. Thrombus aspiration can be effected by activating an aspiration means 38 to draw the occluding thrombus 14 into the aspiration orifice 96 of fluid-assisted microcatheter 104. When aspiration alone is insufficient to remove the thrombus due, for example, to organization of the thrombus, a high pressure fluid delivery means 40 can be used to deliver saline or another biocompatible fluid from a fluid reservoir 42 through fluid delivery intra-luminal space 94, and deliver the pressurized fluid to the fluid jet orifice 92 in the distal end 30 of fluid-assisted microcatheter 104. In this embodiment of the invention, as thrombus 14 is drawn into the aspiration orifice 96, it creates a pressure differential 114 that allows the fluid-assisted microcatheter 104 to maintain communication and control over the thrombus 14. A fluid jet 78 is directed toward the retained thrombus from fluid jet orifice 92. The fluid jet typically operates at between 50 and 600 pounds per square inch, thereby providing sufficient pressure to section off or emulsify the retained thrombus 14 without the risk of distal embolization from thrombetic fragments because the emulsification remains fully contained within the fluid-assisted microcatheter 104. As each section of thrombus is aspirated, new thrombetic material is drawn into the aspiration orifice 96, with the process repeating until all thrombus is removed.

Once all thrombus 14 is removed from the patient vasculature 12, the aspiration means 38 and high pressure fluid delivery means 40 are disabled. After recanalization of the vasculature 12 distal to the occlusion mechanism, (e.g. occlusion balloon 26) a second pressure differential exists between the patient vasculature that is proximal to the occlusion balloon 26 and the patient vasculature that is distal to occlusion balloon 26. The three-way reperfusion valve 33, when used, is turned to the “reperfuse” position to allow a controlled release of blood flow back into the treatment site and equalize the pressure differential around the occlusion balloon 26.

What is claimed is:

1. A neurovascular thrombectomy device comprising:
a) a generally tubular, hollow occlusion microcatheter having a distal end adapted for vascular occlusion and a proximal end adapted for controlling said vascular occlusion; and
b) a generally tubular fluid-assisted microcatheter fitted within said occlusion microcatheter having an open distal end adapted for fluid-assisted thrombus removal and a proximal end adapted for controlling said fluid-assisted thrombus removal.

2. The neurovascular thrombectomy device of claim 1, wherein the fluid-assisted microcatheter is slidably fitted within the occlusion microcatheter.

3. The neurovascular thrombectomy device of claim 1, wherein the fluid-assisted microcatheter is a generally hollow tubular body defining an evacuation path, wherein the hollow body comprises an inner lumen and an outer lumen defining a sealed fluid delivery intra-luminal space.

4. The neurovascular thrombectomy device of claim 3, further comprising:
a) a fluid access port in fluid communication with the fluid delivery intra-luminal space; and
b) a fluid jet orifice disposed near the distal end of the inner lumen.

5. The neurovascular thrombectomy device of claim 4, wherein the fluid-assisted microcatheter delivers fluid under high pressure through the fluid delivery intra-luminal space to the fluid jet orifice and into the evacuation path.

6. The neurovascular thrombectomy device of claim 5, wherein the open distal end of the fluid-assisted microcatheter is perpendicular to the length of the neurovascular thrombectomy device, thereby forming a round aspiration orifice.

7. The neurovascular thrombectomy device of claim 5, wherein the distal end of the fluid-assisted microcatheter is angled 45 degrees relative to the length of the neurovascular thrombectomy device thereby forming an oval aspiration orifice; wherein the fluid jet orifice delivers fluid into the oval aspiration orifice.

8. The neurovascular thrombectomy device of claim 1, wherein the occlusion microcatheter comprises an occlusion balloon.

9. The neurovascular thrombectomy device of claim 8, wherein the occlusion microcatheter comprises an inner tubular member surrounding a working channel and an outer tubular member surrounding the inner tubular member, said outer tubular member and said inner tubular member together defining an inflation intra-luminal space.

10. The neurovascular thrombectomy device of claim 9, wherein the diameter of the working channel is sufficient to slidably accept the fluid-assisted microcatheter.

11. The neurovascular thrombectomy device of claim 9, further comprising an inflation opening disposed on the outer tubular member, wherein the balloon surrounds the inflation opening and is sealably secured to the outer tubular member, and wherein the balloon is in fluid communication with the inflation intra-luminal space through the inflation opening.

12. The neurovascular thrombectomy device of claim 9, wherein the balloon is formed as a contiguous outpocketing of the outer tubular member.

13. The neurovascular thrombectomy device of claim 9, wherein the balloon sealably bridges the outer tubular member and the inner tubular member.

14. The neurovascular thrombectomy device of claim 13, wherein the inner tubular member is longer than and extends distally beyond the outer tubular member.

15. The neurovascular thrombectomy device of claim 1, wherein the occlusion microcatheter comprises a haemostatic seal at an opening in the proximal end of said occlusion catheter.

16. The neurovascular thrombectomy device of claim 1, wherein the proximal end of the occlusion microcatheter adapted for controlling said vascular occlusion comprises a three-way reperfusion valve.

17. The neurovascular thrombectomy device of claim 1, wherein the three-way reperfusion valve is detachably disposed on the proximal end of the proximal end of the occlusion microcatheter adapted for controlling said vascular occlusion.

18. A neurovascular thrombectomy system comprising:
a) the neurovascular thrombectomy device of claim 10;
b) a fluid reservoir connected to the fluid access port;
c) a high pressure fluid delivery means for conveying fluid from the fluid reservoir to the fluid access port;
d) an aspiration means connected to the aspiration exit port; and
e) an aspiration receptacle connected in-line between the aspiration means and the occlusion microcatheter for receiving thrombus and fluid.

19. A method for treating neurovascular thrombus comprising the steps of:
a) accessing a patient blood vessel using a Seldinger technique;
b) inserting a guide wire through the accessed blood vessel and maneuvering the guide wire to a treatment site;
c) placing a guide catheter over the guide wire and advancing the guide catheter to a position slightly proximal to the treatment site;
d) inserting the occlusion microcatheter of the neurovascular thrombectomy system of claim 18 into the guide catheter and advancing the occlusion microcatheter to the treatment site;
e) inserting the fluid-assisted microcatheter into the occlusion microcatheter and sliding the fluid-assisted microcatheter to the treatment site;
f) inflating the occlusion balloon, thereby occluding blood flow proximal to the treatment site;
g) removing the guide wire and capping the guide wire access port
h) simultaneously activating the high pressure fluid delivery means and the aspiration means, thereby powering the fluid-assisted microcatheter of the neurovascular thrombectomy system;
i) advancing the powered fluid-assisted microcatheter to or through a thrombus at the treatment site, thereby removing thrombus;
j) Repeating step i) until all thrombus is removed from the treatment site;
k) simultaneously deactivating the high pressure fluid delivery means and the aspiration means;
l) withdrawing the fluid-assisted microcatheter from the treatment site;
m) restoring blood flow to the treatment site by deflating the occlusion balloon;
n) removing the fluid-assisted microcatheter, occlusion microcatheter and guide catheter from the patient, thereby removing neurovascular thrombus.

20. The method of claim 19, wherein the proximal end of the occlusion microcatheter adapted for controlling said vascular occlusion comprises a three-way reperfusion valve adapted for controllably inflating and deflating the occlusion balloon, wherein steps e) and m) are controlled by the three-way reperfusion valve.
